(19) United States
(12) Patent Application Publication
(10) Pub. No.: US 2014/0358451 A1
(43) Pub. Date: Dec. 4, 2014

(54) Fractional Abundance Estimation from Electrospray Ionization Time-of-Flight Mass Spectrum

(71) Applicants: Sai Buddi, Tempe, AZ (US); Thomas Taylor, Tempe, AZ (US); Randy Nelson, Phoenix, AZ (US); Chad Borges, Avondale, AZ (US); Douglas Cochran, Phoenix, AZ (US)

(72) Inventors: Sai Buddi, Tempe, AZ (US); Thomas Taylor, Tempe, AZ (US); Randy Nelson, Phoenix, AZ (US); Chad Borges, Avondale, AZ (US); Douglas Cochran, Phoenix, AZ (US)

(73) Assignee: ARIZONA BOARD OF REGENTS ON BEHALF OF ARIZONA STATE UNIVERSITY, Scottsdale, AZ (US)

(21) Appl. No.: 14/295,823

(22) Filed: Jun. 4, 2014

Related U.S. Application Data
(60) Provisional application No. 61/831,062, filed on Jun. 4, 2013.

(51) Int. Cl. G01N 33/68; G06F 19/00; G06F 19/10

(52) U.S. Cl. CPC G01N 33/6848; G06F 19/10; G06F 19/34
USPC 702/28

(57) ABSTRACT

Methods and systems for detecting and quantifying signal peaks from ESI-TOF-MS data may include creating a signal model and a noise model for mass spectrometry (MS) data. The method may also include detecting a signal peak based, at least in part, on the signal model and the noise model for the MS data. The method may further include determining an amplitude of the detected signal peak based, at least in part, on the signal model and the noise model for the MS data.

### CROSS-REFERENCE TO RELATED APPLICATIONS

This application claims priority to U.S. Provisional Application No. 61/831,062 filed Jun. 4, 2013, the entire contents of which is specifically incorporated by reference herein without disclaimer.

### FIELD OF THE INVENTION

This disclosure relates to methods and systems for signal processing, and more particularly relates to processing electrospray ionization time-of-flight mass spectrometry (ESI-TOF-MS) data to estimate fractional abundance of biomarkers.

### DESCRIPTION OF RELATED ART

In 2008 the USFDA issued a Guidance of Industry statement, recognizing the conjoined nature of cardiovascular disease (CVD) and type 2 diabetes (T2D) and emphasizing the need to monitor cardiovascular risk during new diabetic drug trials. This led researchers to work towards identifying panels of markers that are able to distinguish the types of CVD in the context of T2D.

Current algorithms that are used for this purpose suffer from numerous drawbacks. For example, current algorithms are not based on sound mathematical concepts, but are instead based on heuristic models, which inherently sacrifice accuracy. Furthermore, current algorithms are too time consuming to process a large number of mass spectra because they require manual input to calculate fractional abundances. Therefore, new methods and algorithms are needed.

### SUMMARY OF THE INVENTION

ESI-TOF-MS data for protein molecules may be modeled and processed to detect and quantify signal peaks from the ESI-TOF-MS data. The fractional abundance of the protein molecules may be estimated from the detected signal peaks to identify protein molecules that may be potential biomarkers to distinguish between CVD and T2D.

In some embodiments, provided is a method for detecting and quantifying signal peaks from ESI-TOF-MS data may include creating a signal model and a noise model for mass spectrometry (MS) data. The method may also include detecting a signal peak based, at least in part, on the signal model and the noise model for the MS data. The method may further include determining an amplitude of the detected signal peak based, at least in part, on the signal model and the noise model for the MS data.

In some embodiments, provided is a computer program product for detecting and quantifying signal peaks from ESI-TOF-MS data may include a non-transitory computer readable medium comprising code for performing the step of creating a signal model and a noise model for mass spectrometry (MS) data. The medium may also include code to perform the steps of detecting a signal peak based, at least in part, on the signal model and the noise model for the MS data, and determining an amplitude of the detected signal peak based, at least in part, on the signal model and the noise model for the MS data.

In some embodiments, provided is an apparatus for detecting and quantifying signal peaks from ESI-TOF-MS data may include a memory and a processor coupled to the memory that is configured to execute the step of creating a signal model and a noise model for mass spectrometry (MS) data. The processor may also execute the steps of detecting a signal peak based, at least in part, on the signal model and the noise model for the MS data, and determining an amplitude of the detected signal peak based, at least in part, on the signal model and the noise model for the MS data.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments.

FIG. 1 is a flow chart illustrating a method for detecting and quantifying signal peaks from ESI-TOF-MS data according to one embodiment of the disclosure.

FIGS. 2A-B are an illustration showing ESI MS peak shapes for two different proteins (FIG. 2A vitamin D binding protein (DBP); FIG. 2B Apolipoprotein C-1 (ApoC1)) at a particular charge state due to isotopic mass spread according to one embodiment of the disclosure.

FIG. 3 is an illustration showing an accelerator region of a TOF assembly according to one embodiment of the disclosure.

FIG. 4 is an illustration showing an abundance estimation for one charge state of HAS according to one embodiment of the disclosure.

### DETAILED DESCRIPTION

Electrospray ionization time-of-flight mass spectrometry (ESI-TOF-MS) may be used to analyze proteins and their variants to discover potential biomarkers for T2D and CVD. In some embodiments, this may include measuring the relative abundances of protein variants in a biological sample of the subject. The proteins may show up as signal peaks embedded in background noise in the mass spectrum, and the abundance of a particular protein is directly related to the area under the signal peak. By mathematically modeling the signal peak shape and noise, these protein signal peaks may be detected in the mass spectrum and an estimate of their amplitude and area may be determined, even when the signal-to-noise ratio is small. A likelihood ratio test may be used for peak signal detection and a maximum likelihood method may be used for amplitude estimation which may then be utilized for area calculation.

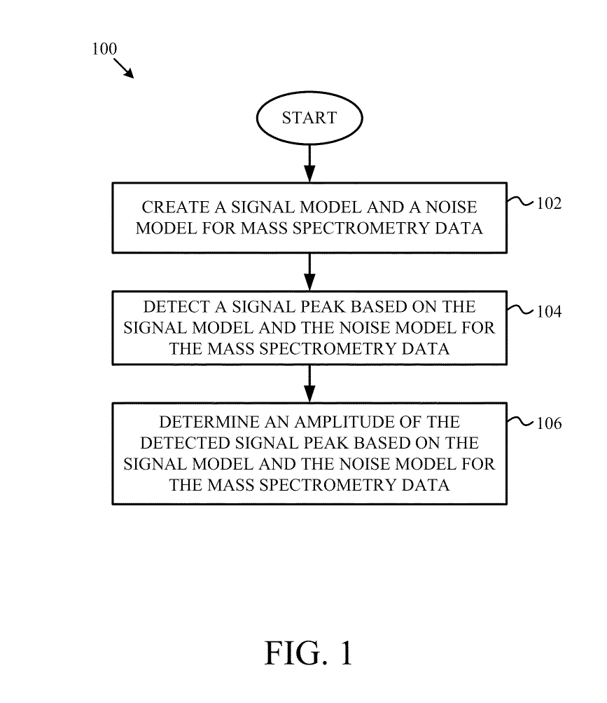

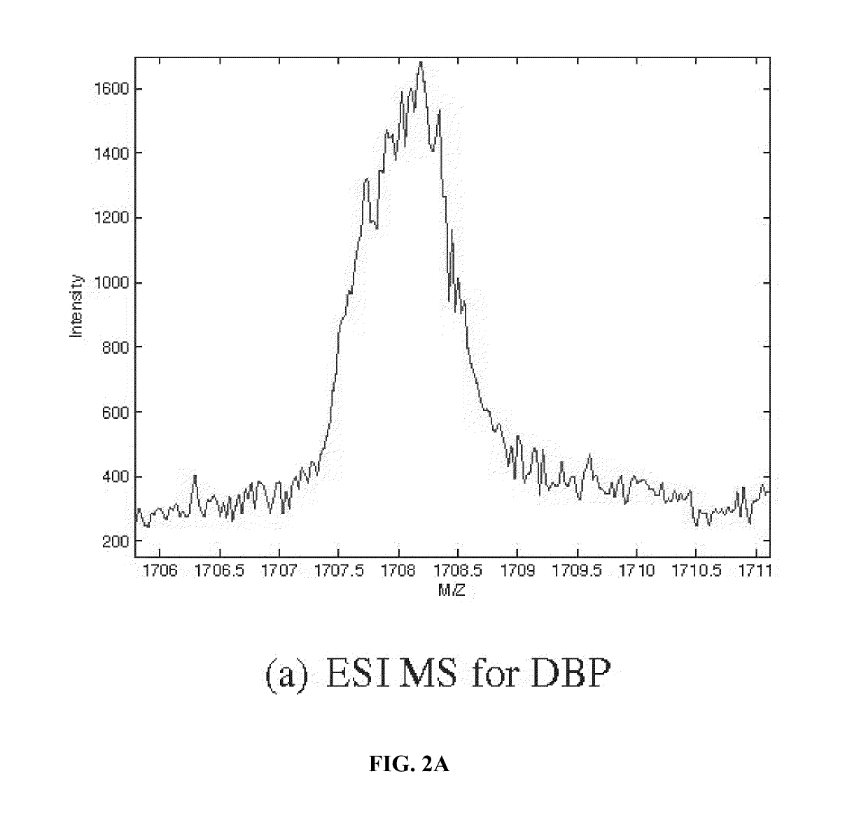

FIG. 1 is a flow chart illustrating a method for detecting and quantifying signal peaks from ESI-TOF-MS data according to one embodiment of the disclosure. The ESI-TOF-MS data may include data for one or more molecular species. The method 100 may begin at block 102 with creating a signal model and a noise model for mass spectrometry data.

In one embodiment, modeling the signal from the ESI-TOF-MS data includes modeling the shape and width of the signal peak. Knowledge of the signal peak shape may be advantageous for numerous mass spectrometry applications. MS equipment physics can be used to derive a theoretical peak shape, and this theoretical shape may be used as is, or adjusted according to empirical measurements of peaks.

In some embodiments, the ability to detect the signal peak and its properties is directly related to the resolution of the signal peak. Resolution is the ability to characterize a signal peak corresponding to any molecular species in the MS in terms of position and shape. Mathematically, resolution for sharp peaks can be defined as the ratio of the mass value and linear width at half the signal height. Signal resolving power may be an important feature when the goal is to distinguish and quantify large protein molecules and their variants present in a sample. ESI may produce multiple charged ions, depending on the size of the molecules, which makes it possible to measure and distinguish large molecular weight proteins at high resolution. The higher resolution usually results in a better mass accuracy. Typically, modern ESI spectrometers achieve resolutions in the order of at least 10⁵. The resolving power of the TOF-MS may depend on the time spread caused by the initial spatial and initial kinetic energy distributions as well as the ability to design the device to reduce that spread. Important parameters that influence the signal resolution include at least the isotopic distribution, the spatial distribution, and the energy distribution of the MS data, as well as the physical limitations of the mass spectrometer used to obtain the MS data. Therefore, a signal model for the ESI-TOF-MS data may provide information about the shape and the width of the signal peak, and may include an isotopic distribution model of the MS data, a spatial distribution model of the MS data, an energy distribution model of the MS data, and a model of physical limitations of a mass spectrometer used to obtain the MS data.

The MS signal for a given molecular species may be modeled by understanding the physics of the instrumentation and mathematically quantifying these limitations. As the effects act independently, the signal shape may be modeled as the convolution of the distributions. Modeling the signal shape as the convolution of the distributions employs first principle calculations based on device physics and molecular properties, which has not been done before. Because isotopic, spatial, and energy distributions degrade the signal resolution, a mathematical model for each provides a sound basis for any peak shape assumptions.

Presence of isotopes is not a limitation of TOF-MS in itself, but they may usually result in degraded resolution. Isotopes may be variants of atoms with different number of neutrons in their nucleus. For example, there are two stable isotopes of carbon that may appear in nature: C¹² and C¹³. They both have the same number of protons (atomic number=6) but C¹³ has an extra neutron and hence a different atomic mass. The natural abundances of the elemental isotopes are known. Molecules contain elemental isotopes according to their natural abundances. The probability of occurrence of these isotopic variants (i.e., the isotopic distribution of a molecule) may be calculated from the atomic composition and the elemental isotope abundances.

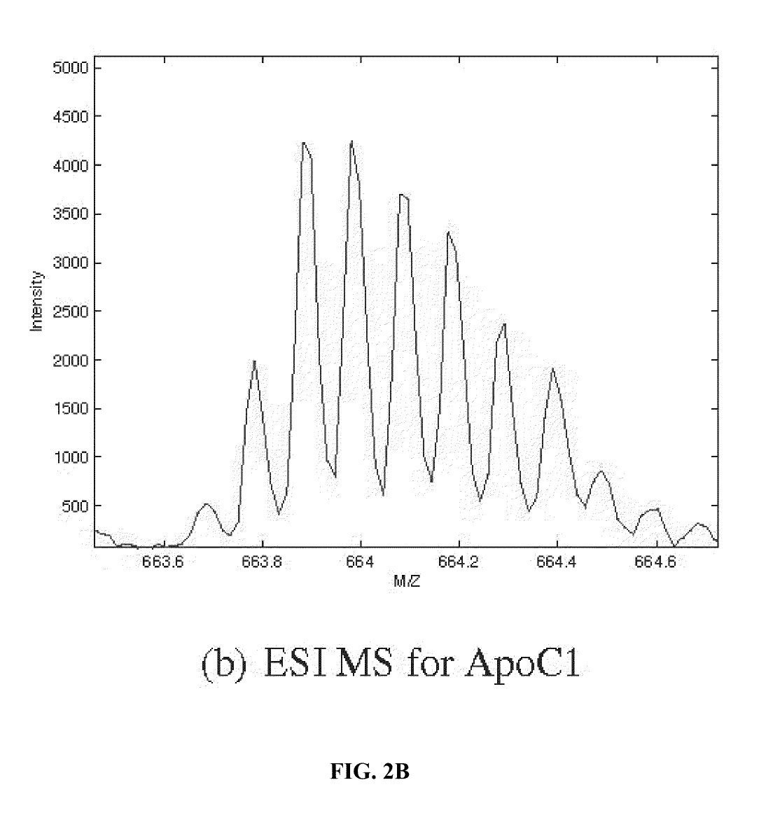

Proteins are usually made of thousands of atoms of H, C, N, O, S, etc. When a protein sample is analyzed in a TOF mass spectrometer, multiple peaks corresponding to the isotopic distribution can exist. If the spectrometer has enough resolution, the isotopic peaks can be resolved, especially for low mass proteins. For the high molecular weight proteins, usually investigated in ESI-TOF-MS, these peaks partially coalesce together resulting in an effective broadened peak. For example, FIG. 2 shows the MS peaks for vitamin D binding protein (DBP) and Apolipoprotein C-1 (ApoC1) for a particular charge state. For the low mass ApoC1, the isotopic peaks are well resolved but only partially resolved for DBP. In some embodiments, the isotopic distribution may be calculated with various stepwise and multinomial expansion techniques that are well known to those of skill in the art. In a particular embodiment, a convolution based method may be used. For example, the method may be based on using the nucleon count, which may be adjusted to the exact mass after calculating the distribution using convolution. Using a convolution based method, the isotopic distribution of a molecule may be the convolution of individual elemental compositions, which can be represented as a product in the Fourier domain. Fast Fourier Transform (FFT) processing can be used for faster computation and efficient memory utilization.

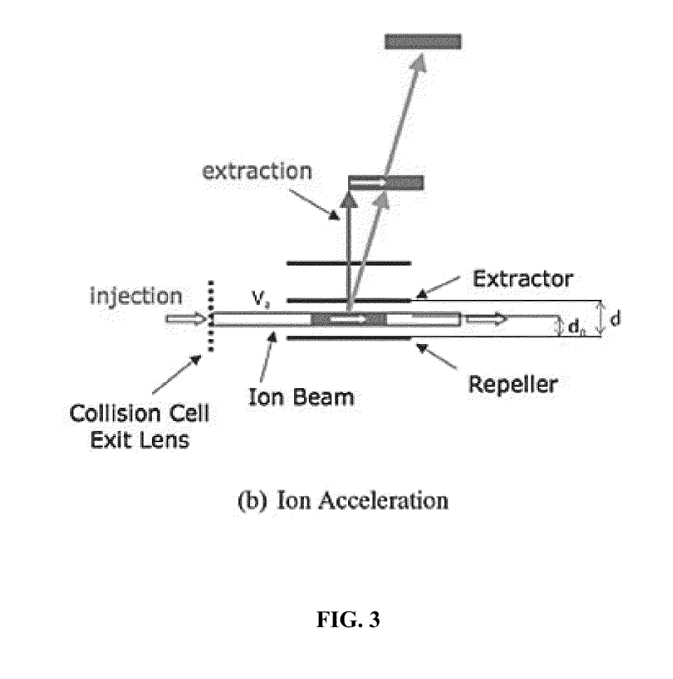

FIG. 3 shows an accelerator region of a TOF assembly according to one embodiment of the disclosure. As seen in FIG. 3, ions may be focused along the axis when entering the orthogonal accelerator region. The spatial distribution of ions between the electrodes may result in a velocity distribution, and thus a TOF distribution, because the ions closer to the repeller plate remain under acceleration longer and hence acquire a larger velocity. The larger the variance in ion spread, the larger the variance in TOF. In one embodiment, the TOF distribution may be modeled with a spatial distribution for the ions. For example, in one embodiment, the time distribution may be written in terms of the spatial distribution as follows:

$$\begin{matrix}{f}_{T} ( t ) = \frac{{f}_{X} ( x )}{\vert {t}^{′} ( x ) \vert} \\ = \frac{{x}^{2} fx ( x )}{\vert {κ}_{2} {x}^{2} - {κ}_{1} \vert}\end{matrix}$$

$$t \geq {t}_{min} > 0$$

with x and fₓ(x) defined as:

$$x = \frac{t - \sqrt{{t}^{2} - 4 {κ}_{1} {κ}_{2}}}{2 {κ}_{2 }}$$

$$and$$

$$\begin{matrix}{f}_{X} ( x ) = \frac{{f}_{D} ( {d}_{0} )}{\vert {x}^{′} \vert} \\ = 2 {xf}_{D} ( {d}_{0} )\end{matrix}$$

$$for$$

$$x \geq 0$$

Similar to spatial distribution, ions may also enter the accelerator region with different velocities and thus kinetic energies. The energy variance of ions may be minimized by controlling the temperature and lens voltage. Therefore, in one embodiment, the time distribution due to the energy spread may be:

f_{ε}(t)˜N(0,Δt²)

Regarding the physical limitations of the mass spectrometer used to obtain the MS data, the multi-channel place (MCP) at a detector may work by a secondary electron multiplication effect in the channel, which may cause the current signal to spread. Depending on the penetration depth of the ion in the channel, the trigger time for the electron avalanche may be different. These effects along with the finite scan rate of ADC may limit the time resolution in MS-TOF. The pulse shape based on the physical limitations may be Gaussian. The signal may be modeled with models available for the isotopic, spatial, and energy distributions, as well as the physical limitations of mass spectrometry components.

From these considerations, a theoretical description of the shape of an MS peak may be developed, which may match the experimentally observed peak shape. This developed peak shape can be used for enhanced detection and amplitude estimation of low amplitude peaks using signal processing methodologies, in contrast to conventional methods which use an assumed peak shape.

In some embodiments, the noise of the ESI-TOF-MS data may also be modeled, as shown at block 102 of FIG. 1. Noise is introduced into the MS signal due to various reasons. For example, the noise can be classified into chemical noise, shot noise, and Johnson noise. Chemical noise may be one of the most significant sources of background distortion in ESI, and may be caused by the presence of solvent molecules. Solvent induced clusters are major contributors to the chemical noise in ESI. Even though the solvent is more volatile than the analyte, it is difficult to get rid of all solvent molecules by use of a heating gas or other such methods, as they often form clusters with each other and with analyte molecules. Sometimes there are chemical impurities in the sample or in the MS equipment itself. These clusters and impurities get charged and traverse the flight path to reach the detector, together with the analyte molecules of interest. Impurities usually give rise to fixed pattern noise at specific m/z values, but the solvent clusters may cause interference in virtually all m/z values. Fragmentation of the analyte or any of these various molecules at any step of the TOF-MS process may add to the complex nature of chemical noise. In some embodiments, careful experiments may be carried out by controlling threshold parameters of the device. For example, solvent cluster chemical noise in MS may be observed by running only solvent through the instrument at operating threshold levels. Goodness-of-fit tests known to those of skill in the art may be used to estimate the probability density with a fair degree of certainty.

One source of noise in the detector arises from the dark current of the MCP. Thermal emission of electrons from in the channels of the MCP gives rise to an avalanche effect, knocking off more electrons along the channel. Under normal operating conditions, the dark current is low but as each frame of MS data is a sum of thousands of spectra, this current adds up. A frame is a snapshot of the sum spectra, generally over 1 second. Hence, if the MS analysis is carried out for 10 minutes, there may be approximately 600 frames in the data set. A chromatogram is the plot of the total intensity (or mass) in the frames over time. The threshold is a negative noise suppression voltage (VTN) applied at the anode of the detector. Dark current can be analyzed by MS analysis without any solvent or sample. The mean, median, and variance may be calculated per frame, using all intensity values in a frame. This gives a sense of the mean noise intensity and variance, which may not be inferred from the chromatogram plot alone. Each point in a chromatogram plot may be the sum of all noise intensities in that frame. The mean, median, and standard deviation plots (calculated from the intensities in each frame) may show that the noise intensities are consistent throughout the frames. Typically, dark current consists of current pulses that are at a lower level than current pulses associated with analyte molecules. These are almost entirely rejected by setting a bias at the detector higher than dark current pulses.

A probability distribution model may statistically describe the chemical noise in ESI-TOF-MS and enable development of a suitable statistical signal processing algorithm. A test of goodness of fit (GOF) may be used to evaluate the agreement between the distribution of these sets of observations and a theoretical distribution. Several such tests are described in statistical literature. For example, the Kolmogorov Smirnov test (KS), the two-sample KS test, the Cramer-von Mises criterion (CM), and the two-sample CM criterion are well-known GOF assessments. In one embodiment, the tests may show that a Gamma distribution may be a suitable model for chemical noise in ESI-TOF-MS data.

In addition to the noise and the signal, ESI-TOF-MS data may include a baseline component, which is not flat, that causes the spectrum to appear to be sitting on top of a time varying baseline. The baseline is generally attributed to chemical noise and detector saturation leading to a slowly decaying charge. In some embodiments, a shifting window algorithm may be used to estimate the baseline. Because a non-flat baseline is prominent when an analyte is present, it is safe to assume that the analyte molecules somehow contribute to this phenomenon. In some embodiments, intact analyte molecules may form the signal peak whereas fragmented and solvated molecules are on the left and right of the signal peak. In general, the baseline may be the result of the sum of all the decaying signals from all the neighboring charge states. In some embodiments, the baseline may be included as part of the signal model, while the intrinsic variability in the baseline may be attributable to “shot noise.” In other embodiments it may be included separately. In many embodiments, the baseline may be considered to be a distortion and therefore included as part of the noise. Therefore, in general, ESI-TOF-MS data may, in some embodiments, be interpreted as data that includes signal, noise, and baseline information. In such an embodiment, the signal peak may turn out to be Gaussian, and the noise may turn out to follow a Gamma distribution.

An extended notion of molecular peak may lump together the sharp peak with coincident “chemical noise” from fragmented analyte and analyte+solvent clusters in a sharp peak super-imposed on slow roll off form. This notion recognizes that the baseline may be comprised of the superposition of the individual peak roll-offs, which may have predictable shape and statistics. This information can be used for enhanced detection and amplitude estimation of low amplitude peaks using signal processing methodologies, in contrast to conventional methods which only recognize the sharp portion of the peak. A characterization of noise, which may be unpredictable or uncontrolled signal variability, as an intrinsic part of the creation of extended peaks may also be developed, and as a consequence a statistical characterization of the noise environment in which low amplitude extended peaks are found. This information can be used for enhanced detection and amplitude estimation of low amplitude peaks using signal processing methodologies, in contrast to conventional methods which model variability as extrinsic to the peak formation process.

Returning to FIG. 1, method 100 may proceed to block 104, where a signal peak may be detected based on the signal model and the noise model for the mass spectrometry data. In some embodiment, the signal peak may be detected with a likelihood ratio test detector that is based on the Neyman-Pearson (NP) lemma. In another embodiment, a log-likelihood ratio test may be used to detect the signal peak. In yet another embodiment, a general likelihood ratio test may be used to detect the signal peak.

At block 106, an amplitude of the detected signal peak may be determined based on the signal model and the noise model for the mass spectrometry data. In one embodiment, a maximum likelihood estimator (MLE) algorithm may be used to estimate the amplitude. The MLE may be used to obtain practical estimates of unknown parameters. The MLE of a parameter may be defined as the value of the parameter that maximizes the likelihood function for a fixed observation. This maximum may be performed over the range of the parameter by differentiating the likelihood function.

Based on the determined amplitude of a signal peak, a fractional abundance of the protein molecule under investigation may be estimated. Because ESI-TOF-MS is not overwhelmingly impacted by fragmentation of macro molecules, such as proteins, ESI-TOF-MS may be useful for biomarker analysis, which may make use of the estimated fractional abundances of protein molecules. Furthermore, in some embodiments, ESI produces multiple charged ions resulting in a low mass-to-charge ratio and thus a higher resolving power. For identifying biomarkers, it may be important to estimate the relative abundance of all the protein variants present in a sample. The signal peak intensity may be a measure of the number of molecules hitting the detector, and the area under the peak may be considered to be a fair measure of the abundance of a molecule in the sample being tested. The abundance of a species may be proportional to the sum of areas of each charge state belonging to the species or the area under the signal peak. Using some of the disclosed embodiments for signal peak detection and signal peak amplitude determination, the area under relevant peaks may be estimated. For example, the fractional abundance of a protein molecule may be estimated by automatically calculating the area under the signal peak for each frame in the chromatogram. In one embodiment, raw data from every relevant frame from a chromatogram may be used for peak detection and estimation. According to an embodiment, the frames where most of the sample molecules are reaching the detector may be chosen as the frames from which the signal peak may be detected and its amplitude estimated.

According to an embodiment, atomic composition and a range of possible charge states of the molecular species may be the only input required for the abundance calculation routine, although other inputs may also be allowed. Furthermore, there may be no pre-processing steps involved and no need for a deconvolution routine according to some embodiments of the disclosure for abundance estimation. Using the isotopic distribution of the molecule, the peak width may be estimated for each charge state. The isotopic distribution provides the theoretical peak location in the MS. Often the MS frames are mis-aligned and the actual peak location may shift along the m/z axis. Hence, the peak detection algorithm may be used to search over a window around the theoretical location. The maximum detector output, when greater than the threshold, may be the location of the peak in the MS. The estimated amplitude at that location may then be used to calculate the area under curve (AUC) of that charge state for that species. The AUC may therefore be calculated from the theoretical peak instead of an MS peak. This way the noise may not accounted for in the abundance calculations.

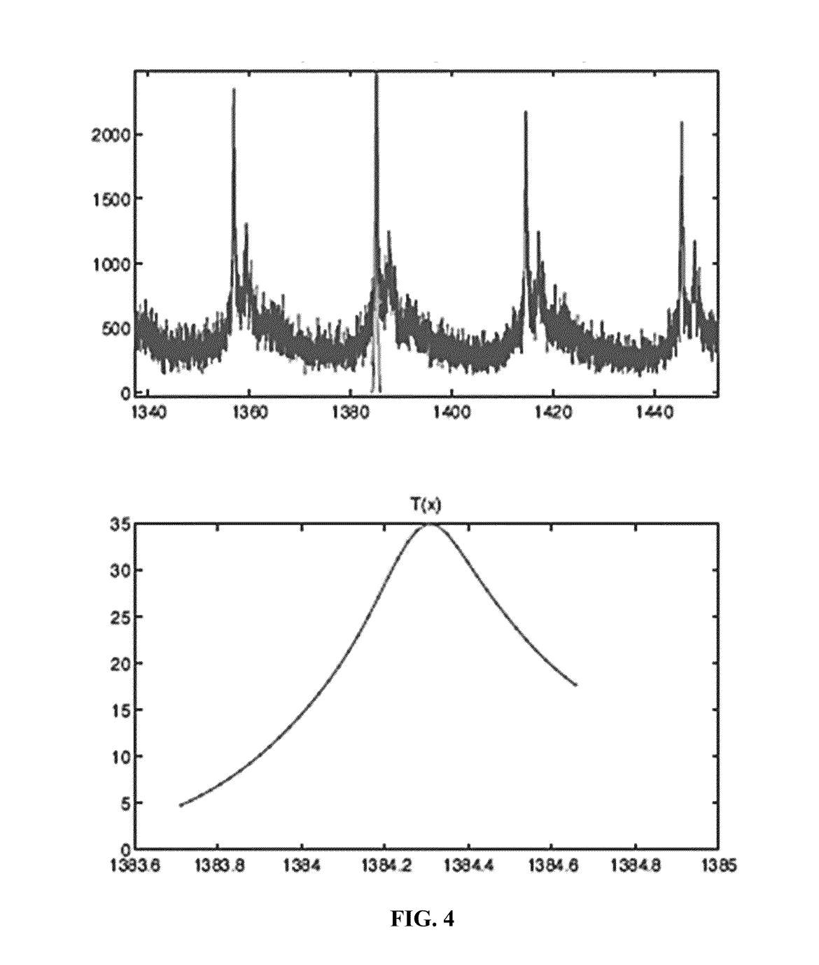

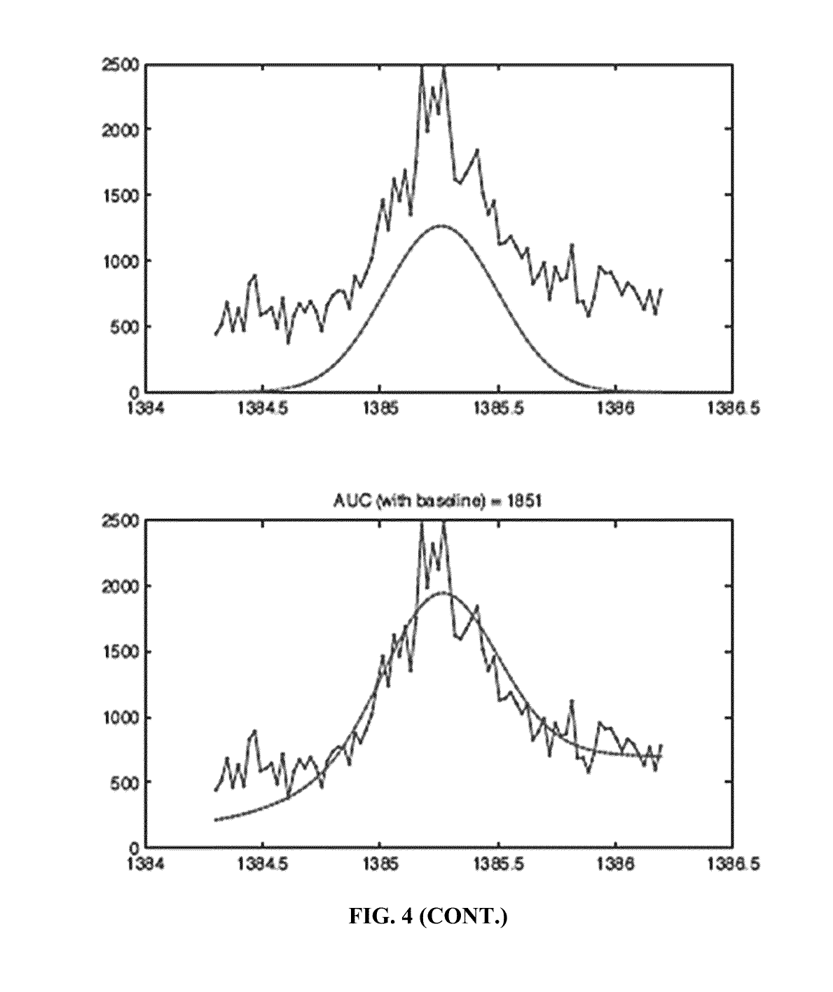

FIG. 4 shows the result of such a detection and estimation method applied to a single frame and charge state of HSA. The top-left figure shows the raw MS data with the theoretical location of the modeled peak for the particular charge state. The detection algorithm may be used to locate the actual location of the peak within a window of the theoretical location, as seen in the bottom-left figure. The top-right figure shows the estimated amplitude and the location of the peak. As the noise model may include the baseline, the estimated amplitude is significantly smaller than the peak, so a baseline correction routine may be unnecessary. The bottom-right figure shows the effect of adding the baseline to the estimated peak. These plots show that the baseline line may be automatically eliminated before abundance calculation is carried out. This step may be repeated for all the charge states and selected frames for a given molecular species.

The abundance of a molecular species may be measured in terms of its AUC, in which the abundance of a molecular species may be the sum of the AUCs of all charge state peaks in all the frames. The relative abundance may be the ratio of the area of targeted species over the total area of all different molecular species present in the sample. In an example presented, HSA may be the primary molecular species and Cysteinylated HSA (Cys-HSA) may be one of its molecular variants. Cys-HSA has a larger molecular weight due to the addition of the Cysteine residue attached via a disulfide bond, and the net molecular mass change may be a 120 Da increase compared to the intact HSA molecule.

In addition to other advantageous features, particular advantageous of the invention disclosed herein may the lack of any pre- or post-processing requirement of the raw MS data. As the noise and baseline may be built into the model, the algorithm may estimate the fractional abundance due to the signal part only. There may also be no need for any MS alignment methods to align all the frames with respect to a reference peak because the detection algorithm may search for the signal within a window of the theoretical location, which is estimated from the isotopic distribution of the molecular species. In addition, fewer number of input parameters may be needed compared to the conventional methods. Moreover, by adjusting a threshold, a user can choose between signal peaks that go into the fractional abundance estimation.

According to an embodiment, a biomarker protein molecule may be identified based, at least in part, on the estimated fractional abundance of protein molecules. As an example of biomarker analysis, consider that diabetes is a chronic disease that has reached epidemic proportions in the US. It is caused by high level of blood sugar, otherwise called hyperglycemia. Most people with diabetes have either type 1 or type 2 diabetes. Type 1 diabetes results from lack of insulin, a hormone that regulates the level of glucose in blood. Type 2 diabetes (T2D) results from insulin resistance of the cell thus making insulin less effective in regulating glucose. Traditionally, T2D is diagnosed by measuring the absolute concentration of glucose. The level of glucose is also reflected by hemoglobin, an oxygen carrying protein in the blood. Hemoglobin undergoes glycation, defined as the bonding of a protein and a sugar molecule, when it is exposed to glucose. This is also one form of post translational modification (PTM) of the protein. Increased levels of glycated hemoglobin (HbA1c) in the blood is an indicator of hyperglycemia. This has led to the acceptance of HbA1c as a marker for diabetes.

The lack of insulin in type 1 diabetes means that insulin therapy is the only effective treatment. Type 2 diabetes has a wider range of therapies available. However, diabetes has been associated with an elevated risk of CVD, the leading cause of mortality among the patients. Hence the T2D therapies have to be evaluated for their effect on cardiovascular risks.

According to one embodiment, a support vector machine may be developed and used for biomarker classification, which may aid in identifying a biomarker protein molecule that distinguishes between a first patient group associated with type 2 diabetes and a history of cardiovascular disease and a second patient group associated with type 2 diabetes and no history of cardiovascular disease. For example, a fractional abundance estimation may be performed to identify a biomarker protein molecule, and using the support vector machine the biomarker may be classified such that it distinguishes between a first patient group associated with type 2 diabetes and a history of cardiovascular disease and a second patient group associated with type 2 diabetes and no history of cardiovascular disease.

SVMs may be binary classifiers by default, but some datasets may have more than two groups to be classified. The binary SVM may also be extended to a multiclass case (MSVM) in some embodiments. The two general strategies may be to either to solve the multiclass case by solving a series of binary problems, or to consider all the classes at once. In methods that use the first strategy, each classifier distinguishes between (a) one of the classes to the rest (one-versus-all) or (b) between every pair of classes (one-versus-one). In one-versus-all case, classification of a test data is done by a winner-takes-all strategy, in which the classifier with the highest output function assigns the class. For the one-versus-one approach, classification is done by a max wins voting strategy, where the class with most votes (from each binary classifier) determines the result.

In some embodiments, there may not be enough data to estimate the likelihood probability for all possible values. In such instances, Monte Carlo methods can be used to simulate data from the available statistics. Even with enough data, sometimes a particular sequences may be more abundant and others less so. This results in the need for estimating the likelihood probability of an object that has never been seen before. Good-Turing methods may be useful in estimating these probabilities. According to an embodiment, a Good-Turing (SGT) method may use a straight line to smooth the regions of inaccurate probability estimates.

If implemented in firmware and/or software, the functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present invention, disclosure, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method, comprising:
creating a signal model and a noise model for mass spectrometry (MS) data;
detecting a signal peak based, at least in part, on the signal model and the noise model for the MS data; and
determining an amplitude of the detected signal peak based, at least in part, on the signal model and the noise model for the MS data.

2. The method of claim 1, further comprising estimating a fractional abundance of a protein molecule based, at least in part, on the determined amplitude of the signal peak.

3. The method of claim 2, further comprising identifying a biomarker protein molecule based, at least in part, on the estimated fractional abundance of the protein molecule.

4. The method of claim 3, wherein the identified biomarker distinguishes between a first patient group associated with type 2 diabetes and a history of cardiovascular disease and a second patient group associated with type 2 diabetes and no history of cardiovascular disease.

5. The method of claim 2, wherein estimating the fractional abundance of the protein molecule comprises automatically calculating the area under the signal peak for each frame in a chromatogram.

6. The method of claim 1, wherein the MS data comprises electrospray ionization time-of-flight mass spectrometry (ESI-TOF-MS) data for at least one molecular species.

7. The method of claim 1, wherein the signal model provides information about a shape and a width of the signal peak and comprises at least one of:
an isotopic distribution model of the MS data;
a spatial distribution model of the MS data;
an energy distribution model of the MS data; and
a model of physical limitations of a mass spectrometer used to obtain the MS data.

8. The method of claim 1, further comprising:
detecting the signal peak with a likelihood ratio test; and
determining the amplitude of the signal peak with a maximum likelihood algorithm.

9. A computer program product, comprising:
a non-transitory computer readable medium comprising code for performing the steps of:
creating a signal model and a noise model for mass spectrometry (MS) data;
detecting a signal peak based, at least in part, on the signal model and the noise model for the MS data; and
determining an amplitude of the detected signal peak based, at least in part, on the signal model and the noise model for the MS data.

10. The computer program product of claim 9, wherein the medium further comprises code for performing the step of estimating a fractional abundance of a protein molecule based, at least in part, on the determined amplitude of the signal peak.

11. The computer program product of claim 10, wherein the medium further comprises code for performing the step of identifying a biomarker protein molecule based, at least in part, on the estimated fractional abundance of the protein molecule.

12. The computer program product of claim 11, wherein the identified biomarker distinguishes between a first patient group associated with type 2 diabetes and a history of cardiovascular disease and a second patient group associated with type 2 diabetes and no history of cardiovascular disease.

13. The computer program product of claim 10, wherein estimating the fractional abundance of the protein molecule comprises automatically calculating the area under the signal peak for each frame in a chromatogram.

14. The computer program product of claim 9, wherein the MS data comprises electrospray ionization time-of-flight mass spectrometry (ESI-TOF-MS) data for at least one molecular species.

15. The computer program product of claim 9, wherein the signal model provides information about a shape and a width of the signal peak and comprises at least one of:
an isotopic distribution model of the MS data;
a spatial distribution model of the MS data;
an energy distribution model of the MS data; and
a model of physical limitations of a mass spectrometer used to obtain the MS data.

16. The computer program product of claim 9, wherein the medium further comprises code for performing the steps of:
detecting the signal peak with a likelihood ratio test; and
determining the amplitude of the signal peak with a maximum likelihood algorithm.

17. An apparatus, comprising:
a memory; and
a processor coupled to the memory, wherein the processor is configured to execute the steps of:
creating a signal model and a noise model for mass spectrometry (MS) data;
detecting a signal peak based, at least in part, on the signal model and the noise model for the MS data; and
determining an amplitude of the detected signal peak based, at least in part, on the signal model and the noise model for the MS data.

18. The apparatus of claim 17, wherein the processor is further configured to execute the step of estimating a fractional abundance of a protein molecule based, at least in part, on the determined amplitude of the signal peak.

19. The apparatus of claim 18, wherein the processor is further configured to execute the step of identifying a biomarker protein molecule based, at least in part, on the estimated fractional abundance of the protein molecule.

20. The apparatus of claim 18, wherein the identified biomarker distinguishes between a first patient group associated with type 2 diabetes and a history of cardiovascular disease and a second patient group associated with type 2 diabetes and no history of cardiovascular disease.

21. The apparatus of claim 18, wherein estimating the fractional abundance of the protein molecule comprises automatically calculating the area under the signal peak for each frame in a chromatogram.

22. The apparatus of claim 17, wherein the MS data comprises electrospray ionization time-of-flight mass spectrometry (ESI-TOF-MS) data for at least one molecular species.

23. The apparatus of claim 17, wherein the signal model provides information about a shape and a width of the signal peak and comprises at least one of:
an isotopic distribution model of the MS data;
a spatial distribution model of the MS data;
an energy distribution model of the MS data; and
a model of physical limitations of a mass spectrometer used to obtain the MS data.

24. The apparatus of claim 17, wherein the processor is further configured to execute the steps of:
detecting the signal peak with a likelihood ratio test; and
determining the amplitude of the signal peak with a maximum likelihood algorithm.
